(19) United States
(12) Patent Application Publication
(10) Pub. No.: US 2020/0309733 A1
(43) Pub. Date: Oct. 1, 2020

(54) SENSOR ELEMENT FOR GAS SENSOR

(71) Applicant: NGK INSULATORS, LTD., Nagoya-shi (JP)

(72) Inventor: Takashi HINO, Kasugai-shi (JP)

(21) Appl. No.: 16/827,759

(22) Filed: Mar. 24, 2020

(30) Foreign Application Priority Data
Mar. 29, 2019 (JP) 2019-066786

(51) Int. Cl. G01N 27/407; G01N 33/00

(52) U.S. Cl. CPC G01N 27/4077; G01N 27/4071; G01N 33/0037

(57) ABSTRACT

A gas sensor element includes: an element base being a ceramic structure including a sensing part to sense a gas component to be measured; and a leading-end protective layer being a porous layer disposed around an outer periphery of the element base in a predetermined range from an end portion of the element base on a side of the sensing part. A near-surface portion of the leading-end protective layer near a surface thereof has a porosity of 15% to 30%, and has a value of surface roughness Ra of 3 μm to 35 μm.

### CROSS-REFERENCE TO RELATED APPLICATION

The present application claims priority from Japanese application JP 2019-066786, filed on Mar. 29, 2019, the contents of which is hereby incorporated by reference into this application.

### BACKGROUND OF THE INVENTION

### Field of the Invention

The present invention relates to a sensor element for a gas sensor, and, in particular, to a surface protective layer thereof.

### Description of the Background Art

As a gas sensor for determining concentration of a desired gas component contained in a measurement gas, such as an exhaust gas from an internal combustion engine, a gas sensor that includes a sensor element made of an oxygen-ion conductive solid electrolyte, such as zirconia (ZrO₂), and including some electrodes on the surface and the inside thereof has been widely known. As the sensor element, a sensor element having an elongated planar shape, including a protective layer formed of a porous body (porous protective layer) in an end portion in which a part for introducing the measurement gas is provided, and further including a surface protective layer having a smaller porosity than the porous protective layer outside the porous protective layer has been known (see Japanese Patent No. 5387555, for example).

The protective layer is provided to the surface of the sensor element to secure water resistance of the sensor element when the gas sensor is in use. Specifically, the protective layer is provided to prevent water-induced cracking of the sensor element under the action of thermal shock caused by heat (cold) from water droplets adhering to the surface of the sensor element.

In a gas sensor disclosed in Japanese Patent No. 5387555, a surface protective layer is made water repellent at a high temperature (500° C. or more) utilizing the Leidenfrost phenomenon to repel water droplets adhering to a sensor element to thereby prevent water-induced cracking of the sensor element.

More particularly, Japanese Patent No. 5387555 discloses that the surface protective layer preferably has surface roughness (arithmetic mean roughness) Ra of 3 μm or less to surely develop the Leidenfrost phenomenon to thereby sufficiently maintain water repellency of the surface protective layer at a high temperature, and water repellency cannot sufficiently be secured when the surface protective layer has surface roughness Ra of more than 3 μm. In Japanese Patent No. 5387555, the amount of water exposure of 10 μL is determined as a reference value for water-induced cracking (water resistance).

### SUMMARY

The present invention relates to a sensor element for a gas sensor, and is, in particular, directed to a configuration of a surface protective layer thereof.

It is found, from intensive studies made by the inventors of the present invention, that, when the surface protective layer has arithmetic mean roughness Ra meeting a predetermined requirement, thermal shock acting on the surface protective layer when water droplets adhere to the surface protective layer can be suppressed more compared with a case where the Leidenfrost phenomenon is developed, and thermal shock resistance of the surface protective layer can be secured without positively developing the Leidenfrost phenomenon to thereby secure water resistance of the sensor element.

According to the present invention, a sensor element for a gas sensor includes: an element base being a ceramic structure including a sensing part to sense a gas component to be measured; and a leading-end protective layer being a porous layer disposed around an outer periphery of the element base in a predetermined range from an end portion of the element base on a side of the sensing part, wherein a near-surface portion of the leading-end protective layer near a surface thereof has a porosity of 15% to 30%, and has a value of surface roughness Ra of 3 μm to 35 μm.

Accordingly, a sensor element in which thermal shock acting on the surface of the leading-end protective layer when water droplets adhere to the surface is suppressed is thereby achieved.

Preferably, the near-surface portion covers an inner portion of the leading-end protective layer having a porosity of 30% to 90% and having lower thermal conductivity than the near-surface portion.

In this case, a sensor element in which thermal shock resistance of the leading-end protective layer as a whole is increased to thereby secure water resistance is achieved.

It is thus an object of the present invention to provide a sensor element in which thermal shock acting on the surface protective layer when water droplets adhere to the surface protective layer is suppressed, and, further, thermal shock resistance of the surface protective layer can be secured without positively developing the Leidenfrost phenomenon to thereby secure water resistance.

These and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a schematic external perspective view of a sensor element 10;

FIG. 2 is a schematic view illustrating a configuration of a gas sensor 100 including a sectional view taken along a longitudinal direction of the sensor element 10;

FIGS. 3A and 3B schematically illustrate states when water droplets adhere to surfaces of two leading-end protective layers being in a high temperature state and having different values of surface roughness; and

FIG. 4 is a flowchart of processing at the manufacture of the sensor element 10.

### DESCRIPTION OF THE PREFERRED EMBODIMENTS

<Overview of Sensor Element and Gas Sensor>

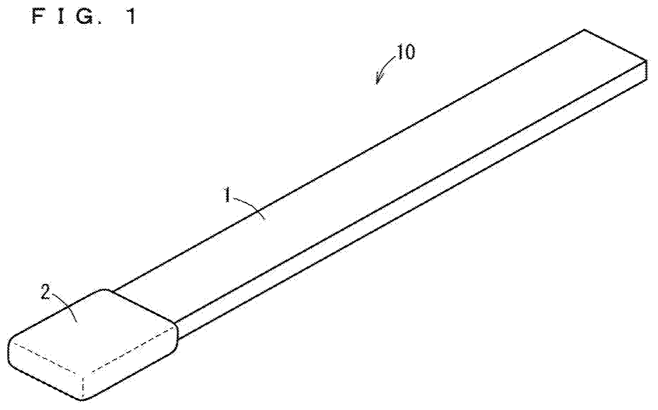

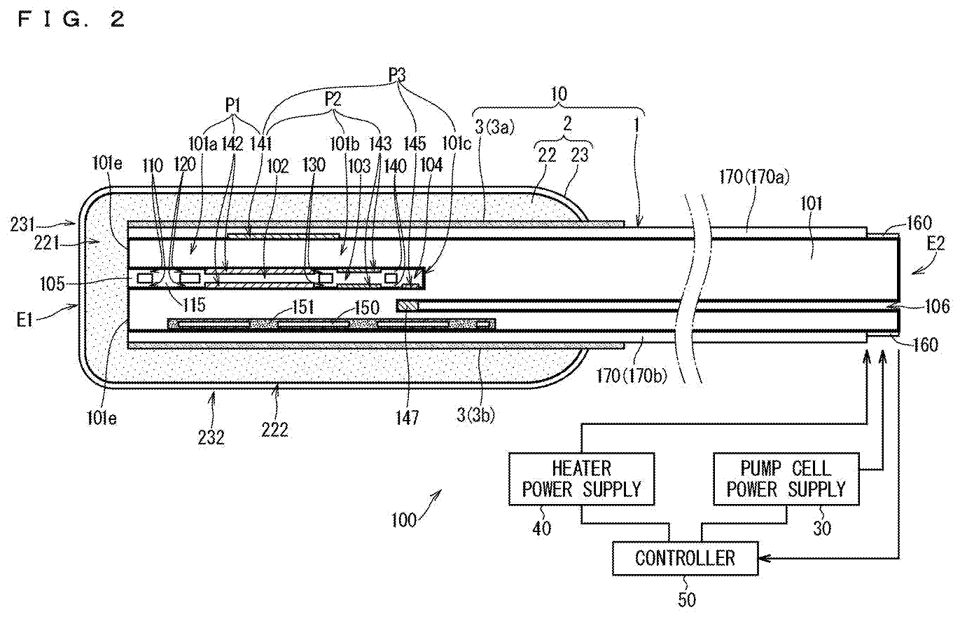

FIG. 1 is a schematic external perspective view of a sensor element (gas sensor element) 10 according to an embodiment of the present invention. FIG. 2 is a schematic view illustrating a configuration of a gas sensor 100 including a sectional view taken along a longitudinal direction of the sensor element 10. The sensor element 10 is a ceramic structure as a main component of the gas sensor 100 sensing a predetermined gas component in a measurement gas, and measuring concentration thereof. The sensor element 10 is a so-called limiting current gas sensor element.

In addition to the sensor element 10, the gas sensor 100 mainly includes a pump cell power supply 30, a heater power supply 40, and a controller 50.

As illustrated in FIG. 1, the sensor element 10 has a configuration in which one end portion of an elongated planar element base 1 is covered with a porous leading-end protective layer 2.

As illustrated in FIG. 2, the element base 1 includes an elongated planar ceramic body 101 as a main structure, main surface protective layers 170 are provided on two main surfaces of the ceramic body 101, and, in the sensor element 10, the leading-end protective layer 2 is further provided outside both an end surface (a leading end surface 101e of the ceramic body 101) and four side surfaces on one leading end portion. The four side surfaces other than opposite end surfaces in the longitudinal direction of the sensor element 10 (or the element base 1, or the ceramic body 101) are hereinafter simply referred to as side surfaces of the sensor element 10 (or the element base 1, or the ceramic body 101).

The ceramic body 101 is made of ceramic containing, as a main component, zirconia (yttria stabilized zirconia), which is an oxygen-ion conductive solid electrolyte. Various components of the sensor element 10 are provided outside and inside the ceramic body 101. The ceramic body 101 having the configuration is dense and airtight. The configuration of the sensor element 10 illustrated in FIG. 2 is just an example, and a specific configuration of the sensor element 10 is not limited to this configuration.

The sensor element 10 illustrated in FIG. 2 is a so-called serial three-chamber structure type gas sensor element including a first internal chamber 102, a second internal chamber 103, and a third internal chamber 104 inside the ceramic body 101. That is to say, in the sensor element 10, the first internal chamber 102 communicates, through a first diffusion control part 110 and a second diffusion control part 120, with a gas inlet 105 opening to the outside on a side of one end portion E1 of the ceramic body 101 (to be precise, communicating with the outside through the leading-end protective layer 2), the second internal chamber 103 communicates with the first internal chamber 102 through a third diffusion control part 130, and the third internal chamber 104 communicates with the second internal chamber 103 through a fourth diffusion control part 140. A path from the gas inlet 105 to the third internal chamber 104 is also referred to as a gas distribution part. In the sensor element 10 according to the present embodiment, the distribution part is provided straight along the longitudinal direction of the ceramic body 101.

The first diffusion control part 110, the second diffusion control part 120, the third diffusion control part 130, and the fourth diffusion control part 140 are each provided as two slits vertically arranged in FIG. 2. The first diffusion control part 110, the second diffusion control part 120, the third diffusion control part 130, and the fourth diffusion control part 140 provide predetermined diffusion resistance to a measurement gas passing therethrough. A buffer space 115 having an effect of buffering pulsation of the measurement gas is provided between the first diffusion control part 110 and the second diffusion control part 120.

An outer pump electrode 141 is provided on an outer surface of the ceramic body 101, and an inner pump electrode 142 is provided in the first internal chamber 102. Furthermore, an auxiliary pump electrode 143 is provided in the second internal chamber 103, and a measurement electrode 145 as a sensing part to directly sense a gas component to be measured is provided in the third internal chamber 104. In addition, a reference gas inlet 106 which communicates with the outside and through which a reference gas is introduced is provided on a side of the other end portion E2 of the ceramic body 101, and a reference electrode 147 is provided in the reference gas inlet 106.

In a case where a target of measurement of the sensor element 10 is NOx in the measurement gas, for example, concentration of a NOx gas in the measurement gas is calculated by a process as described below.

First, the measurement gas introduced into the first internal chamber 102 is adjusted to have a substantially constant oxygen concentration by a pumping action (pumping in or out of oxygen) of a main pump cell P1, and then introduced into the second internal chamber 103. The main pump cell P1 is an electrochemical pump cell including the outer pump electrode 141, the inner pump electrode 142, and a ceramic layer 101a that is a portion of the ceramic body 101 existing between these electrodes. In the second internal chamber 103, oxygen in the measurement gas is pumped out of the element by a pumping action of an auxiliary pump cell P2 that is also an electrochemical pump cell, so that the measurement gas is in a sufficiently low oxygen partial pressure state. The auxiliary pump cell P2 includes the outer pump electrode 141, the auxiliary pump electrode 143, and a ceramic layer 101b that is a portion of the ceramic body 101 existing between these electrodes.

The outer pump electrode 141, the inner pump electrode 142, and the auxiliary pump electrode 143 are each formed as a porous cermet electrode (e.g., a cermet electrode made of ZrO₂ and Pt that contains Au of 1%). The inner pump electrode 142 and the auxiliary pump electrode 143 to be in contact with the measurement gas are each formed using a material having weakened or no reducing ability with respect to a NOx component in the measurement gas.

NOx in the measurement gas caused by the auxiliary pump cell P2 to be in the low oxygen partial pressure state is introduced into the third internal chamber 104, and reduced or decomposed by the measurement electrode 145 provided in the third internal chamber 104. The measurement electrode 145 is a porous cermet electrode also functioning as a NOx reduction catalyst that reduces NOx existing in an atmosphere in the third internal chamber 104. During the reduction or decomposition, a potential difference between the measurement electrode 145 and the reference electrode 147 is maintained constant. Oxygen ions generated by the above-mentioned reduction or decomposition are pumped out of the element by a measurement pump cell P3. The measurement pump cell P3 includes the outer pump electrode 141, the measurement electrode 145, and a ceramic layer 101c that is a portion of the ceramic body 101 existing between these electrodes. The measurement pump cell P3 is an electrochemical pump cell pumping out oxygen generated by decomposition of NOx in an atmosphere around the measurement electrode 145.

Pumping (pumping in or out of oxygen) of the main pump cell P1, the auxiliary pump cell P2, and the measurement pump cell P3 is achieved, under control performed by the controller 50, by the pump cell power supply (variable power supply) 30 applying a voltage necessary for pumping across electrodes included in each of the pump cells. In a case of the measurement pump cell P3, a voltage is applied across the outer pump electrode 141 and the measurement electrode 145 so that the potential difference between the measurement electrode 145 and the reference electrode 147 is maintained at a predetermined value. The pump cell power supply 30 is typically provided for each pump cell.

The controller 50 detects a pump current Ip2 flowing between the measurement electrode 145 and the outer pump electrode 141 in accordance with the amount of oxygen pumped out by the measurement pump cell P3, and calculates a NOx concentration in the measurement gas based on a linear relationship between a current value (NOx signal) of the pump current Ip2 and the concentration of decomposed NOx.

The gas sensor 100 preferably includes a plurality of electrochemical sensor cells, which are not illustrated, sensing the potential difference between each pump electrode and the reference electrode 147, and each pump cell is controlled by the controller 50 based on a signal detected by each sensor cell.

In the sensor element 10, a heater 150 is buried in the ceramic body 101. The heater 150 is provided, below the gas distribution part in FIG. 2, over a range from the vicinity of the one end portion E1 to at least a location of formation of the measurement electrode 145 and the reference electrode 147. The heater 150 is provided mainly to heat the sensor element 10 to enhance oxygen-ion conductivity of the solid electrolyte forming the ceramic body 101 when the sensor element 10 is in use. More particularly, the heater 150 is provided to be surrounded by an insulating layer 151.

The heater 150 is a resistance heating body made, for example, of platinum. The heater 150 generates heat by being powered from the heater power supply 40 under control performed by the controller 50.

The sensor element 10 according to the present embodiment is heated by the heater 150 when being in use so that the temperature at least in a range from the first internal chamber 102 to the second internal chamber 103 becomes 500° C. or more. In some cases, the sensor element 10 is heated so that the temperature of the gas distribution part as a whole from the gas inlet 105 to the third internal chamber 104 becomes 500° C. or more. These are to enhance the oxygen-ion conductivity of the solid electrolyte forming each pump cell and to desirably demonstrate the ability of each pump cell. In this case, the temperature in the vicinity of the first internal chamber 102, which becomes the highest temperature, becomes approximately 700° C. to 800° C.

In the following description, from among the two main surfaces of the ceramic body 101, a main surface (or an outer surface of the sensor element 10 having the main surface) which is located on an upper side in FIG. 2 and on a side where the main pump cell P1, the auxiliary pump cell P2, and the measurement pump cell P3 are mainly provided is also referred to as a pump surface, and a main surface (or an outer surface of the sensor element 10 having the main surface) which is located on a lower side in FIG. 2 and on a side where the heater 150 is provided is also referred to as a heater surface. In other words, the pump surface is a main surface closer to the gas inlet 105, the three internal chambers, and the pump cells than to the heater 150, and the heater surface is a main surface closer to the heater 150 than to the gas inlet 105, the three internal chambers, and the pump cells.

A plurality of electrode terminals 160 are formed on the respective main surfaces of the ceramic body 101 on the side of the other end portion E2 to establish electrical connection between the sensor element 10 and the outside. These electrode terminals 160 are electrically connected to the above-mentioned five electrodes, opposite ends of the heater 150, and a lead for detecting heater resistance, which is not illustrated, through leads provided inside the ceramic body 101, which are not illustrated, to have a predetermined correspondence relationship. Application of a voltage from the pump cell power supply 30 to each pump cell of the sensor element 10 and heating by the heater 150 by being powered from the heater power supply 40 are thus performed through the electrode terminals 160.

The sensor element 10 further includes the above-mentioned main surface protective layers 170 (170a and 170b) on the pump surface and the heater surface of the ceramic body 101. The main surface protective layers 170 are layers made of alumina, having a thickness of approximately 5 μm to 30 μm, and including pores with a porosity of approximately 20% to 40%, and are provided to prevent adherence of any foreign matter and poisoning substances to the main surfaces (the pump surface and the heater surface) of the ceramic body 101 and the outer pump electrode 141 provided on the pump surface. The main surface protective layer 170a on the pump surface thus functions as a pump electrode protective layer for protecting the outer pump electrode 141.

In the present embodiment, the porosity is obtained by applying a known image processing method (e.g., binarization processing) to a scanning electron microscope (SEM) image of an evaluation target.

The main surface protective layers 170 are provided over substantially all of the pump surface and the heater surface except that the electrode terminals 160 are partially exposed in FIG. 2, but this is just an example. The main surface protective layers 170 may locally be provided in the vicinity of the outer pump electrode 141 on the side of the one end portion E1 compared with the case illustrated in FIG. 2.

<Details of Leading-End Protective Layer>

In the sensor element 10, the leading-end protective layer 2 is provided around an outermost periphery of the element base 1 having a configuration as described above in a predetermined range from the one end portion E1.

The leading-end protective layer 2 is provided in a manner of surrounding a portion of the element base 1 in which the temperature becomes high (up to approximately 700° C. to 800° C.) when the gas sensor 100 is in use, in order to secure water resistance in the portion to thereby suppress the occurrence of cracking (water-induced cracking) of the element base 1 due to thermal shock caused by local temperature reduction upon direct exposure of the portion to water.

In addition, the leading-end protective layer 2 is provided to secure poisoning resistance to prevent poisoning substances, such as Mg, from entering into the sensor element 10.

As illustrated in FIG. 2, in the sensor element 10 according to the present embodiment, the leading-end protective layer 2 includes two layers: an inner leading-end protective layer 22 and an outer leading-end protective layer 23. An underlying layer 3 is provided between the leading-end protective layer 2 (the inner leading-end protective layer 22) and the element base 1.

The underlying layer 3 is a layer provided to secure bonding (adhesion) of the inner leading-end protective layer 22 formed thereon (further the outer leading-end protective layer 23). The underlying layer 3 is provided at least on two main surfaces of the element base 1 on a side of the pump surface and a side of the heater surface. That is to say, the underlying layer 3 includes an underlying layer 3a on the side of the pump surface and an underlying layer 3b on the side of the heater surface. The underlying layer 3, however, is not provided on a side of the leading end surface 101e of the ceramic body 101 (of the element base 1).

The underlying layer 3 is made of alumina, has a porosity of 30% to 60%, and has a thickness of 15 μm to 50 μm. In contrast to the inner leading-end protective layer 22 and the outer leading-end protective layer 23, the underlying layer 3 is formed along with the element base 1 in a process of manufacturing the element base 1 as described below.

The inner leading-end protective layer 22 and the outer leading-end protective layer 23 are provided in this order from inside to cover the leading end surface 101e and the four side surfaces on the side of the one leading end portion E1 of the element base 1 (around an outer periphery of the element base 1 on the side of the one leading end portion E1). A portion of the inner leading-end protective layer 22 on the side of the leading end surface 101e is particularly referred to as a leading-end portion 221, and a portion of the inner leading-end protective layer 22 on the side of the pump surface and the side of the heater surface is particularly referred to as a main surface portion 222. Similarly, a portion of the outer leading-end protective layer 23 on the side of the leading end surface 101e is particularly referred to as a leading-end portion 231, and a portion of the outer leading-end protective layer 23 on the side of the pump surface and the side of the heater surface is particularly referred to as a main surface portion 232. The main surface portion 222 of the inner leading-end protective layer 22 is adjacent to the underlying layer 3.

The inner leading-end protective layer 22 is made of alumina, has a porosity of 30% to 90%, and has a thickness of 200 μm to 1000 μm. The outer leading-end protective layer 23 is made of alumina, has a porosity of 10% to 30%, and has a thickness of 50 μm to 300 μm. The leading-end protective layer 2 thereby has a configuration in which the inner leading-end protective layer 22 having lower thermal conductivity than the outer leading-end protective layer 23 is covered with the outer leading-end protective layer 23 having a smaller porosity than the inner leading-end protective layer 22. The inner leading-end protective layer 22 is provided as a layer having low thermal conductivity to have a function to suppress heat conduction from the outside to the element base 1.

In addition, the outer leading-end protective layer 23 is provided so that the surface thereof serving as the surface of the leading-end protective layer 2 as a whole has a value of arithmetic mean roughness (hereinafter, simply referred to as mean roughness) Ra of 3 μm to 35 μm. This is to suppress development of the Leidenfrost phenomenon on the surface of leading-end protective layer 2 at a time when the sensor element 10 is in use and the temperature thereof becomes up to 700° C. to 800° C. Such a value of the surface roughness Ra, however, contradicts conventional technology (e.g., technology disclosed in Japanese Patent No. 5387555) in which a sensor element is made water repellent utilizing the Leidenfrost phenomenon to solve the problem of water-induced cracking of the sensor element, and the surface roughness Ra is required to be set to 3 μm or less to surely develop the Leidenfrost phenomenon.

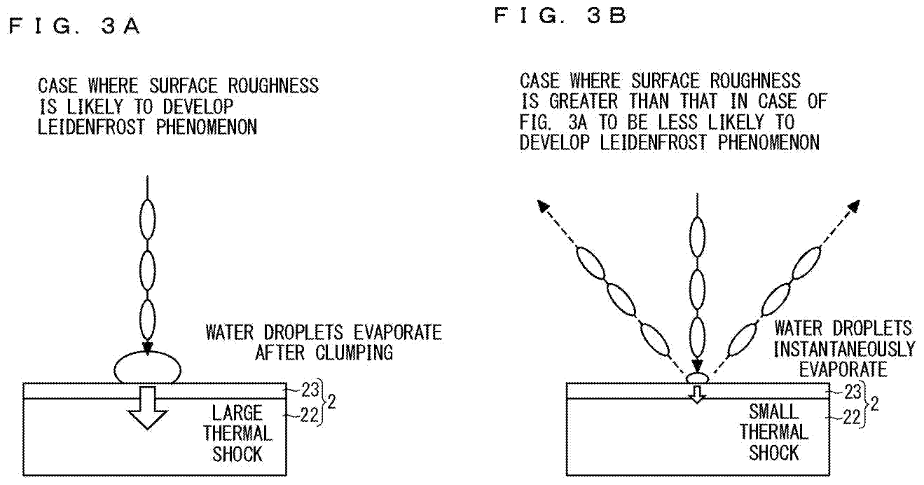

From intensive studies made by the inventors of the present invention, however, it is found that water resistance can be secured even in a case where the outer leading-end protective layer 23 is provided to have the above-mentioned value of the surface roughness Ra of 3 μm to 35 μm in order to suppress development of the Leidenfrost phenomenon. In order to explain the finding, FIGS. 3A and 3B schematically illustrate states when water droplets adhere to surfaces of two leading-end protective layers each being in a high temperature state but having different values of surface roughness.

FIG. 3A illustrates the state when the outer leading-end protective layer 23 has a value of the surface roughness Ra suitable for development of the Leidenfrost phenomenon. When the value of the surface roughness is suitable for development of the Leidenfrost phenomenon, the shape having many recesses (particularly reentrant cavities), which are likely to generate bubbles upon contact with liquid at a high temperature, is predominant.

In this case, water droplets going to adhere to the surface of the outer leading-end protective layer 23 are put into a state of being repelled by the surface due to the Leidenfrost phenomenon (more particularly, in a state of being floated by water vapor generated between the water droplets and the surface of the outer leading-end protective layer 23 due to film boiling caused by generation of bubbles below the water droplets), and water droplets in such a state are likely to clump together before evaporating. A large water droplet is thus likely to be formed with the progress of clumping in a case illustrated in FIG. 3A. A water droplet increased in size due to clumping, however, becomes difficult to maintain surface tension thereof to collapse soon afterward, so that the Leidenfrost phenomenon developed in the water droplet disappears. Then, moisture forming the water droplet comes into direct contact with the surface of the outer leading-end protective layer 23 in the high temperature state, and, at the contact, thermal shock acts on the inside of the leading-end protective layer 2. Thermal shock in this case increases with increasing size of the water droplet before collapse.

On the other hand, FIG. 3B illustrates the state when the outer leading-end protective layer 23 has a greater value of the surface roughness Ra than that in the case illustrated in FIG. 3A. In this case, the surface of the outer leading-end protective layer 23 has greater roughness than that in the state suitable for development of the Leidenfrost phenomenon, and bubbles are less likely to be generated upon contact with liquid at a high temperature. Water droplets adhering to the surface of the outer leading-end protective layer 23 thus instantaneously evaporate due to nucleate boiling before being repelled due to the Leidenfrost phenomenon and thereby clumped. In this case, thermal shock to which the outer leading-end protective layer 23 is subjected by adherence of water droplets is smaller than thermal shock caused when water droplets increased in size by clumping collapse in the case illustrated in FIG. 3A. This means that the outer leading-end protective layer 23 serving as the surface of the leading-end protective layer 2 has greater thermal shock resistance to cold caused by adherence of water droplets in the case illustrated in FIG. 3B than in the case illustrated in FIG. 3A. The sensor element 10 including the leading-end protective layer 2 having such great thermal shock resistance is a sensor element having great water resistance in which water-induced cracking caused by thermal shock is suitably suppressed.

In the present embodiment, in view of the above-mentioned findings, the outer leading-end protective layer 23 serving as the surface of the leading-end protective layer 2 is provided to have a porosity of 15% to 30%, and have a value of the surface roughness Ra of 3 μm to 35 μm, so that development of the Leidenfrost phenomenon on the surface of the leading-end protective layer 2 when the sensor element 10 is heated to a high temperature is suppressed to thereby suppress thermal shock acting on the surface of the leading-end protective layer 2. In addition, the outer leading-end protective layer 23 surrounds the inner leading-end protective layer 22 having a porosity of 30% to 90% and having low thermal conductivity in order to increase thermal shock resistance to cold of the leading-end protective layer 2 as a whole, thereby to secure water resistance of the sensor element 10.

From another perspective, this means that thermal shock acting on the surface of the leading-end protective layer 2 is suppressed when a near-surface portion of the leading-end protective layer 2 is formed to have a porosity of 15% to 30% and a value of the surface roughness Ra of 3 μm to 35 μm, and, further, when the leading-end protective layer 2 is configured so that the near-surface portion covers an inner portion having a porosity of 30% to 90% and having low thermal conductivity, thermal shock resistance of the leading-end protective layer 2 is increased as a whole and water resistance of the sensor element 10 is secured.

Thus, as long as at least the near-surface portion of the leading-end protective layer 2 has a similar porosity and similar surface roughness to the outer leading-end protective layer 23, and a portion having a similar porosity to the inner leading-end protective layer 22 is present inside the leading-end protective layer 2 to have a similar thickness to the inner leading-end protective layer 22, the inner leading-end protective layer 22 and the outer leading-end protective layer 23 are not necessarily required to be clearly separated from each other along the thickness of the leading-end protective layer 2, and a change in form between these portions may be transitional.

The inner leading-end protective layer 22 and the outer leading-end protective layer 23 are formed by sequentially thermal spraying (plasma-spraying) materials for them with respect to the element base 1 having a surface on which the underlying layer 3 has been formed. This is to develop an anchoring effect between the inner leading-end protective layer 22 and the underlying layer 3 formed in advance in the process of manufacturing the element base 1 to thereby secure bonding (adhesion) of the inner leading-end protective layer 22 (including the outer leading-end protective layer 23 formed outside the inner leading-end protective layer 22) to the underlying layer 3. In other words, this means that the underlying layer 3 has a function to secure bonding (adhesion) of the inner leading-end protective layer 22. Secured bonding (adhesion) in this manner suppresses development of the Leidenfrost phenomenon on the surface of the leading-end protective layer 2, and thus, separation of the leading-end protective layer 2 from the element base 1 caused by thermal shock caused when water droplets adhere to the surface is suitably suppressed.

The inner leading-end protective layer 22 and the outer leading-end protective layer 23 are provided not to cover the underlying layer 3 (3a and 3b) as a whole but to expose an end portion of the underlying layer 3 on a side opposite the side of the one end portion E1 in the longitudinal direction of the sensor element 10. This is to more surely secure bonding (adhesion) of the inner leading-end protective layer 22 (including the outer leading-end protective layer 23 formed outside the inner leading-end protective layer 22) to the underlying layer 3.

As described above, in the sensor element 10 according to the present embodiment, the near-surface portion of the leading-end protective layer 2 is provided to have a porosity of 15% to 30%, and have a value of the surface roughness Ra of 3 μm to 35 μm, so that thermal shock acting on the surface of the leading-end protective layer 2 when water droplets adhere is suppressed. Furthermore, the leading-end protective layer 2 is configured so that the near-surface portion covers the inner portion having a porosity of 30% to 90% and having low thermal conductivity to increase thermal shock resistance of the leading-end protective layer 2 as a whole, to thereby secure water resistance of the sensor element 10.

<Process of Manufacturing Sensor Element>

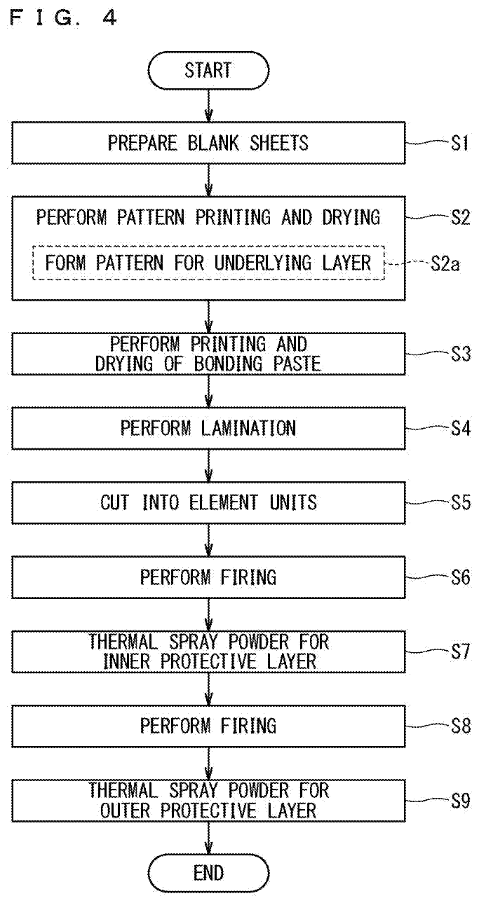

One example of a process of manufacturing the sensor element 10 having a configuration and features as described above will be described next. FIG. 4 is a flowchart of processing at the manufacture of the sensor element 10.

At the manufacture of the element base 1, a plurality of blank sheets (not illustrated) being green sheets containing the oxygen-ion conductive solid electrolyte, such as zirconia, as a ceramic component and having no pattern formed thereon are prepared first (step S1).

The blank sheets have a plurality of sheet holes used for positioning in printing and lamination. The sheet holes are formed to the blank sheets in advance prior to pattern formation through, for example, punching by a punching machine. Green sheets corresponding to a portion of the ceramic body 101 in which an internal space is formed also include penetrating portions corresponding to the internal space formed in advance through, for example, punching as described above. The blank sheets are not required to have the same thickness, and may have different thicknesses in accordance with corresponding portions of the element base 1 eventually formed.

After preparation of the blank sheets corresponding to the respective layers, pattern printing and drying are performed on the individual blank sheets (step S2). Specifically, a pattern of various electrodes, a pattern of the heater 150 and the insulating layer 151, a pattern of the electrode terminals 160, a pattern of the main surface protective layers 170, a pattern of internal wiring, which is not illustrated, and the like are formed. Application or placement of a sublimable material (vanishing material) for forming the first diffusion control part 110, the second diffusion control part 120, the third diffusion control part 130, and the fourth diffusion control part 140 is also performed at the time of pattern printing. In addition, a pattern to form the underlying layer 3 (3a and 3b) is printed onto blank sheets to become an uppermost layer and a lowermost layer after lamination (step S2a).

The patterns are printed by applying pastes for pattern formation prepared in accordance with the properties required for respective formation targets onto the blank sheets using known screen printing technology. At formation of the underlying layer 3, for example, an alumina paste that can form the underlying layer 3 having a desired porosity and thickness in the sensor element 10 eventually obtained is used. A known drying means can be used for drying after printing.

After pattern printing on each of the blank sheets, printing and drying of a bonding paste are performed to laminate and bond the green sheets (step S3). The known screen printing technology can be used for printing of the bonding paste, and the known drying means can be used for drying after printing.

The green sheets to which an adhesive has been applied are then stacked in a predetermined order, and the stacked green sheets are crimped under predetermined temperature and pressure conditions to thereby form a laminated body (step S4). Specifically, crimping is performed by stacking and holding the green sheets as a target of lamination on a predetermined lamination jig, which is not illustrated, while positioning the green sheets at the sheet holes, and then heating and pressurizing the green sheets together with the lamination jig using a lamination machine, such as a known hydraulic pressing machine. The pressure, temperature, and time for heating and pressurizing depend on a lamination machine to be used, and these conditions may be determined appropriately to achieve good lamination. The pattern to form the underlying layer 3 may be formed on the laminated body obtained in this manner.

After the laminated body is obtained as described above, the laminated body is cut out at a plurality of locations to obtain unit bodies eventually becoming the individual element bases 1 (step S5).

The unit bodies as obtained are then each fired at a firing temperature of approximately 1300° C. to 1500° C. (step S6). The element base 1 having main surfaces on which the underlying layer 3 is provided is thereby manufactured. That is to say, the element base 1 is generated by integrally firing the ceramic body 101 made of the solid electrolyte, the electrodes, and the main surface protective layers 170 along with the underlying layer 3. Integral firing is performed in this manner, so that the electrodes each have sufficient adhesion strength in the element base 1.

After the element base 1 is manufactured in the above-mentioned manner, the inner leading-end protective layer 22 and the outer leading-end protective layer 23 are formed with respect to the element base 1. The inner leading-end protective layer 22 is formed by thermal spraying powder (alumina powder) for forming the inner leading-end protective layer prepared in advance at a location of the element base 1 as a target of formation of the inner leading-end protective layer 22 to have an intended thickness (step S7), and then firing the element base 1 on which an applied film has been formed in the above manner (step S8). The alumina powder for forming the inner leading-end protective layer contains alumina powder having predetermined particle size distribution and a pore-forming material at a ratio corresponding to a desired porosity, and the pore-forming material is pyrolyzed by firing the element base 1 after thermal spraying to suitably form the inner leading-end protective layer 22 having a high porosity of 30% to 90%. Known technology is applicable to thermal spraying and firing.

Upon formation of the inner leading-end protective layer 22, powder (alumina powder) for forming the outer leading-end protective layer similarly prepared in advance and containing alumina powder having predetermined particle size distribution is thermal sprayed at a location of the element base 1 as a target of formation of the outer leading-end protective layer 23 to have an intended thickness (step S9) to thereby form the outer leading-end protective layer 23 having a desired porosity. The alumina powder for forming the outer leading-end protective layer does not contain the pore-forming material. Known technology is also applicable to the thermal spraying.

The sensor element 10 is obtained by the above-mentioned procedures. The sensor element 10 thus obtained is housed in a predetermined housing, and built into the body (not illustrated) of the gas sensor 100.

<Modifications>

The above-mentioned embodiment is targeted at a sensor element having three internal chambers, but the sensor element is not necessarily required to have a three-chamber structure. That is to say, the sensor element may have one internal chamber or two internal chambers.

### Examples

Seven types of sensor elements 10 (Samples No. 1 to No. 7) having different combinations of the porosity of the inner leading-end protective layer (hereinafter, an inner layer) 22, the porosity of the outer leading-end protective layer (hereinafter, an outer layer) 23, and surface roughness Ra of the outer layer (i.e., of the leading-end protective layer 2) were manufactured. The inner layers 22 and the outer layers 23 were each made of alumina. The inner layers 22 each had an intended thickness of 600 μm, and the outer layers 23 each had an intended thickness of 200 μm. The outer layers 23 were manufactured under different plasma conditions at thermal spraying to have different degrees of surface roughness while being manufactured using the same alumina powder and having a porosity of 20% in samples other than the sample No. 2 (the sample No. 2 has a porosity of 15%).

The surface roughness Ra of the surface on the side of the pump surface of the outer layer 23 as one surface of the leading-end protective layer 2 was obtained for each of the sensor elements 10. Specifically, roughness in a transverse direction of the element at a total of 30 locations different in the longitudinal direction of the element was measured using a roughness measuring machine VR3200 by Keyence Corporation, and values were obtained for individual results of measurement of roughness as obtained.

Three types of sensor elements 10 (Samples No. 8 to 10) each not including the outer layer 23 and having different combinations of the intended thickness and the porosity of the inner layer 22 were also manufactured for comparison. For each of the sensor elements 10, the surface roughness Ra of the surface on the side of the pump surface of the inner layer 22 as one surface of the leading-end protective layer 2 was obtained in a manner similar to the above-mentioned manner A water resistance test was conducted on each of the sensor elements 10 as obtained. The water resistance test was conducted by applying a water droplet of 0.1 μL at a time to the side of the pump surface of the sensor element 10 while measuring the pump current through the main pump cell P1 in a state of the sensor element 10 being heated by the heater 150 to approximately 500° C. to 900° C., and evaluating the maximum amount of water causing no abnormalities in an output of measurement. In these Examples, the maximum amount of water in the above case is referred to as “water resistance” (in μL). It is considered that any abnormality occurs in the output of measurement in the water resistance test because the leading-end protective layer 2 is subjected to thermal shock to cause cracking of the sensor element 10, so that a value of “water resistance” in these Examples serves as an indicator of how unlikely cracking is to be caused and further as an indicator of thermal shock resistance of the leading-end protective layer 2.

The intended thicknesses (film thicknesses) and the porosities of the respective layers, a range of the value of the surface roughness Ra, and the results of evaluation of water resistance are shown for each of the samples in Table 1 as a list.

TABLE 1

| | INNER LAYER | OUTER LAYER | | | SURFACE | WATER |
|---|---|---|---|---|---|---|
| SAMPLE | FILM THICKNESS | FILM THICKNESS | INNER LAYER | OUTER LAYER | ROUGHNESS | RESISTANCE |
| NO. | (μm) | (μm) | POROSITY (%) | POROSITY (%) | Ra (μm) | (μL) |
| 1 | 600 | 200 | 80 | 20 | 15-26 | 30 |
| 2 | 600 | 200 | 60 | 15 | 8-15 | 29 |
| 3 | 600 | 200 | 60 | 20 | 9-17 | 29 |
| 4 | 600 | 200 | 50 | 20 | 20-35 | 25.6 |
| 5 | 600 | 200 | 40 | 20 | 3-16 | 21.1 |
| 6 | 600 | 200 | 60 | 20 | 35 OR MORE | 9.4 |
| 7 | 600 | 200 | 50 | 20 | 6-15 | 15 |
| 8 | 600 | NOT PROVIDED | 80 | — | CANNOT BE EVALUATED | 10 |
| 9 | 800 | NOT PROVIDED | 50 | — | 20-35 | 9.6 |
| 10 | 800 | NOT PROVIDED | 50 | — | 35 OR MORE | 5 |

In Table 1, the surface roughness Ra in the sample No. 8 is indicated as “CANNOT BE EVALUATED” because surface roughness at most locations of measurement of roughness are too large to be measured, and only the lowest limit is shown and the highest limit is not shown in the range of the surface roughness Ra in each of the samples No. 6 and No. 10 because surface roughness at some locations of measurement of roughness are too large to be measured.

As shown in Table 1, the samples No. 8 to No. 10 each not including the outer layer 23 had the value of water resistance of at most 10 μL although having a large value of the surface roughness Ra of 20 μm or more, whereas, as for the samples each including the outer layer 23, the samples No. 1 to No. 5 and No. 7 had a large value of water resistance of 15 μL or more while only the sample No. 6 having a value of the surface roughness Ra of 35 μm or more had a similar value of water resistance to the samples No. 8 to No. 10.

The results show that, in a case where the sensor element 10 is manufactured to satisfy the condition that, as for the leading-end protective layer 2, the inner layer 22 has a porosity of 30% to 90%, and the outer layer 23 has a porosity of 15% to 30% and has a value of the surface roughness Ra of 3 μm to 35 μm, the sensor element 10 having greater thermal shock resistance of the leading-end protective layer 2, and thus having greater water resistance than that not satisfying the condition can be obtained.

In particular, comparison between the samples No. 4 and No. 9 having similar values of the surface roughness Ra (Ra=20 μm to 35 μm) and comparison between the samples No. 6 and No. 10 having similar values of the surface roughness Ra (Ra≥35 μm) show that the former including the outer layer 23 has a greater value of water resistance in each of the cases. This can be said to suggest that the outer layer 23 having a large value of surface roughness suppresses development of the Leidenfrost phenomenon and thermal shock acting on the surface of the leading-end protective layer 2 when water droplets adhere.

While the invention has been shown and described in detail, the foregoing description is in all aspects illustrative and not restrictive. It is therefore understood that numerous modifications and variations can be devised without departing from the scope of the invention.

What is claimed is:

1. A sensor element for a gas sensor comprising:
an element base being a ceramic structure including a sensing part to sense a gas component to be measured; and
a leading-end protective layer being a porous layer disposed around an outer periphery of said element base in a predetermined range from an end portion of said element base on a side of said sensing part, wherein
a near-surface portion of said leading-end protective layer near a surface thereof has a porosity of 15% to 30%, and has a value of surface roughness Ra of 3 μm to 35 μm.

2. The sensor element according to claim 1, wherein
said near-surface portion covers an inner portion of said leading-end protective layer having a porosity of 30% to 90% and having lower thermal conductivity than said near-surface portion.

3. A sensor element for a gas sensor comprising:
an element base being a ceramic structure including a sensing part to sense a gas component to be measured; and
a leading-end protective layer being a porous layer disposed around an outer periphery of said element base in a predetermined range from an end portion of said element base on a side of said sensing part, wherein
said leading-end protective layer includes, at an outermost periphery thereof, an outer leading-end protective layer having a porosity of 15% to 30% and having a value of surface roughness Ra of 3 μm to 35 μm.

4. The sensor element according to claim 3, wherein
said leading-end protective layer further includes, inside said outer leading-end protective layer, an inner leading-end protective layer having a porosity of 30% to 90% and having lower thermal conductivity than said outer leading-end protective layer.

5. The sensor element according to claim 1, further comprising
an underlying layer disposed at least on two main surface of said element base, wherein
said leading-end protective layer is disposed to cover said end portion and four side surfaces of said element base including said two main surfaces on which said underlying layer is disposed.

6. The sensor element according to claim 2, further comprising
an underlying layer disposed at least on two main surface of said element base, wherein
said leading-end protective layer is disposed to cover said end portion and four side surfaces of said element base including said two main surfaces on which said underlying layer is disposed.

7. The sensor element according to claim 3, further comprising
an underlying layer disposed at least on two main surface of said element base, wherein
said leading-end protective layer is disposed to cover said end portion and four side surfaces of said element base including said two main surfaces on which said underlying layer is disposed.

8. The sensor element according to claim 4, further comprising
an underlying layer disposed at least on two main surface of said element base, wherein
said leading-end protective layer is disposed to cover said end portion and four side surfaces of said element base including said two main surfaces on which said underlying layer is disposed.
